SEQUENCE LISTING (2 sequences)

SEQ ID NO: 1 - DNA, 1790 bp, Homo sapiens
gtgaatctctggggccaggaagaccctgctgcccggaagagcctcatgttccgtgggggctgggcggacatacatatacgggctccaggctgaacggctcgggccacttacacaccactgcctgataaccatgctggctgccacagtcctgaccctggccctgctgggcaatgcccatgcctgctccaaaggcacctcgcacgaggcaggcatcgtgtgccgcatcaccaagcctgccctcctggtgttgaaccacgagactgccaaggtgatccagaccgccttccagcgagccagctacccagatatcacgggcgagaaggccatgatgctccttggccaagtcaagtatgggttgcacaacatccagatcagccacttgtccatcgccagcagccaggtggagctggtggaagccaagtccattgatgtctccattcagaacgtgtctgtggtcttcaaggggaccctgaagtatggctacaccactgcctggtggctgggtattgatcagtccattgacttcgagatcgactctgccattgacctccagatcaacacacagctgacctgtgactctggtagagtgcggaccgatgcccctgactgctacctgtctttccataagctgctcctgcatctccaaggggagcgagagcctgggtggatcaagcagctgttcacaaatttcatctccttcaccctgaagctggtcctgaagggacagatctgcaaagagatcaacgtcatctctaacatcatggccgattttgtccagacaagggctgccagcatcctttcagatggagacattggggtggacatttccctgacaggtgatcccgtcatcacagcctcctacctggagtcccatcacaagggtcatttcatctacaagaatgtctcagaggacctccccctccccaccttctcgcccacactgctgggggactcccgcatgctgtacttctggttctctgagcgagtcttccactcgctggccaaggtagctttccaggatggccgcctcatgctcagcctgatgggagacgagttcaaggcagtgctggagacctggggcttcaacaccaaccaggaaatcttccaagaggttgtcggcggcttccccagccaggcccaagtcaccgtccactgcctcaagatgcccaagatctcctgccaaaacaagggagtcgtggtcaattcttcagtgatggtgaaattcctctttccacgcccagaccagcaacattctgtagcttacacatttgaagaggatatcgtgactaccgtccaggcctcctattctaagaaaaagctcttcttaagcctcttggatttccagattacaccaaagactgtttccaacttgactgagagcagctccgagtccatccagagcttcctgcagtcaatgatcaccgctgtgggcatccctgaggtcatgtctcggctcgaggtagtgtttacagccctcatgaacagcaaaggcgtgagcctcttcgacatcatcaaccctgagattatcactcgagatggcttcctgctgctgcagatggactttggcttccctgagcacctgctggtggatttcctccagagcttgagctagaagtctccaaggaggtcgggatggggcttgtagcagaaggcaagcaccaggctcacagctggaaccctggtgtctcctccagcgtggtggaagttgggttaggagtacggagatggagattggctcccaactcctccctatcctaaaggcccactggcattaaagtgctgtatccaag

SEQ ID NO: 2 - DNA, 1435 bp, Homo sapiens
gaattctgagggcagagcgggccactttctcaggcctctgatttcatactgtggtgttagttacttctgagaggacagcttgcgccagagctctattttttatgttagaggctccttctgcctgcagactctgctgtctgggaagggcacagcgttaggagggagagggaggtgtgagtccctccgtggacccgctgctttgtacttctctatctcatttccttttcagcaccactctgggaaatcagtattccagccccattttatcctcagaaaattgaggctctgagatgttatctctgtgacctgggtcctattacgtgccaaaggcatcatttaagcctaagatgtcctggctccaaggtgtcagcatctggaagacaggcgccctcatcctgccatccctgctgcggcttcactgtgggcccaggggacatctcagccccgagaaggtcagcggcccctcctggaccaccgactccccgcagaactcctctgtgccctctcctcaccagaccttgttcctcccagttgctcccacagccagggggcagtgagggctgctcttcccccagccccactgaggaacccaggaaggtgaacgagagaatcagtcctggtgggggctggggagggcccagacatgagaccagctcctcccccagggatgttatcagtgggtccagagggcaaaatagggagcctggtggagggaggggcaaaggcctcgggctctgagcggccttggcttctccaccaacccctgccctacactcagggggaggcggcggtggggcacacagggtgggggcgggtggggggctgctgggtgagcagcactcgcctgcctggattgaaacccagagatggaggtgctgggaggggctgtgagagctcagccctgtaaccaggccttgcgagccactgatgcccggtcttctgtgcctttactccaaacatcccccagcccaagccacccacttgttctcaagtctgaagaagcccctcacccctctactccaggctgtgttcagggcttggggctggtggagggaggggcctgaaattccagtgtgaaaggctgagatgggcccgacccctggcctatgtccaagccatttcccctctcaccagcctctccctggggagccagtcagctaggaaggaatgaggctccccaggcccacccccagttcctgagctcatctgggctgcagggctggcgggacagcagcgtggactcagtctcctagggatttcccaactctcccgcccgcttgctgcatctggacaccctgcctcaggccctcatctccactggtcagcaggtgacctttgcccagcgccctgggtcctcagtgcctgctgccctggagatgatataaaacaggtcagaaccctcctgcctgtctgctcagttcatccctagaggcag

(19) United States
(12) Patent Application Publication
(10) Pub. No.: US 2003/0140357 A1
(43) Pub. Date: Jul. 24, 2003

(54) Animal models for cardiac disease

(76) Inventors: Victoria L. M. Herrera, Westwood, MA (US); Nelson Ruiz-Opazo, Westwood, MA (US)

Correspondence Address: CLARK & ELBING LLP, 101 FEDERAL STREET, BOSTON, MA, 02110, US

(21) Appl. No.: 10/281,329

(22) Filed: Oct. 24, 2002

Related U.S. Application Data
(63) Continuation of application No. 09/709,079, filed on Nov. 8, 2000, now abandoned.
(60) Provisional application No. 60/164,235, filed on Nov. 8, 1999.

(51) Int. Cl. A01K 67/027

(52) U.S. Cl. 800/9; 800/14

(57) ABSTRACT

The invention features a non-human transgenic mammal that exhibits an atherosclerosis phenotype as a consequence of the expression of a transgene encoding mammalian cholesteryl ester transfer protein (CETP). The transgenic model of the invention is unique in its development of the full spectrum of coronary artery disease that simulates human histopathology.

### CROSS REFERENCE TO RELATED APPLICATIONS

This application claims priority from U.S. provisional application No. 60/164,235, filed Nov. 8, 1999.

### FIELD OF THE INVENTION

The invention relates to the field of cardiac disease.

### BACKGROUND OF THE INVENTION

Epidemiological studies have consistently demonstrated increased risk of coronary artery disease (CHD) and its various clinical manifestations with increasing severity of hypertension¹. Both systolic and diastolic blood pressure levels contribute independently, each associated with a dramatic increase in CHD death rate in every age group, especially in younger men^{1,2} However, mechanisms underlying this interaction have not been elucidated³. Although multiple murine transgenic models of atherosclerosis have been developed, none have addressed the interaction of polygenic hypertension and atherosclerosis. Studies of experimental hypertension in animal models have shown that defined lipid-rich atherosclerotic plaques do not develop in the absence of hyperlipidemia⁴. In hypercholesterolemic Watanabe rabbits, induced renovascular hypertension increased the area and extent of aortic atherosclerotic lesions⁵. However, the impact of hypertension on CHD pathogenesis cannot be deduced from this model since the extent of aortic lesions is not related to survival in the Watanabe rabbit⁶; and that genetic factors for aortic and coronary lesion development are distinct⁷. Transgenic overexpression of human cholesteryl ester transfer protein (hCETP) was selected based on the correlative notion that non-detectable CETP activity contributes to rodent atherosclerosis resistance⁸. This transgenic design would also address current ambiguities on the role of CETP activity in atherosclerosis using a strategic non-murine model system. On the one hand, CETP is thought to play a proatherogenic role since it mediates redistribution of plasma cholesterol from lipoproteins associated with atheroprotection into proatherogenic apolipoprotein B-containing lipoproteins that in turn increase the amount of cholesterol delivered to peripheral cells⁹. On the other hand, CETP could be atheroprotective since CETP activity contributes to the reverse cholesterol transport⁹. In humans, CETP deficiency has been reported to be anti-atherogenic^{10,11}, however generalization of these observations has been questioned¹². Similarly, polar observations have been observed in transgenic mouse models demonstrating CETP-proatherogenicity¹³, impaired remnant and/or LDL clearance-dependent CETP-proatherogenicity¹⁴, as well as atheroprotection in the presence of hypertriglyceridemia¹⁵. Additionally, since single transgenic CETP mouse models exhibit a mild atherosclerotic phenotype restricted to the development of early atherosclerotic lesions and only in response to a diet containing a very high cholesterol content and bile salts¹³, a transgenic CETP-polygenic hypertensive rat model would allow Koch's hypothesis testing. More specifically, if there is a mechanistic basis to the epidemiological observation of worse atherosclerosis in the presence of polygenic (essential) hypertension in humans, transgenic hCETP polygenic hypertensive Dahl S rats should exhibit worse atherosclerosis phenotype than transgenic mouse CETP models.

Transgenic mouse hCETP models exist (transgenic mouse models expressing hCETP), in which hCETP expression is driven by different promoters, for example, the metallothionein promoter or the hCETP promoter. The mouse models did not exhibit a phenotype mimicking human disease, i.e., combined dyslipidemia (high cholesterol, high triglyceride, low HDLc levels). Furthermore, even the most robust mouse transgenic atherosclerosis model (Apolipoprotein E knockout model) does not develop coronary artery lesions that progress to destabilized and thrombosed vulnerable-plaques.

### SUMMARY OF THE INVENTION

Accordingly, the invention features a non-human transgenic mammal that exhibits an atherosclerosis phenotype as a consequence of the expression of a transgene encoding mammalian cholesteryl ester transfer protein (CETP), where the mammal, in its non-transgenic form, is salt-sensitive such that it becomes hypertensive on a diet containing a higher than normal salt content, and where the CETP transgene is expressed under the control of a promoter, e.g., the ApoC3 promoter, that causes the transgene to be expressed in the liver of the mammal. Preferred mammals are rodents, most preferably rats. As is discussed below, a particularly suitable rat is the Dahl S rat, and a preferred CETP gene is the human CETP gene (hCETP).

The animals of the invention are useful as models for human cardiac disease; the animals, like human cardiac patients, develop both atherosclerosis and hypertension, and the atherosclerosis development in the animals, as in humans, involves vulnerable plaque. Thus, the animals can be used to test a compound for its potential efficacy in treating or slowing the development of heart disease, by administering the compound to the animal under conditions under which the animal would, in the absence of treatment, develop atherosclerosis and hypertension; if the compound slows or reverses either or both of these conditions, it is a likely therapeutic candidate for human use.

We have determined that the course of the development of the disease in the animals of the invention mimics the development of cardiac disease in humans. Thus, early, benign—appearing histological changes in the coronary arteries of the animals are likely, in fact, to be early stage signs of disease. These early stage indicators can be used to detect incipient disease in human patients, by observing the counterparts of the changes in human patients. In addition, the identification of cellular features as arterial lesions progress, from early stage lesions, to later, vulnerable plaques, will be important in the development of imaging tools for human vulnerable plaque identification and severity assessment.

In more detail, the animals of the invention develop a coronary artery disease spectrum that simulates the histopathological features detected in post-mortem human vulnerable plaques and plaque destabilization (unstable plaque). The histological features associated with human vulnerable plaques that are detected in the animal lesions are: a) thin fibrous caps, b) lipid richness>40% of lesion area, c) paucity of smooth muscle cells, d) eccentric, non-occlusive, e) active inflammation, and f) foam cell enriched.

The associated features of human vulnerable plaque destabilization that are detected in the animal lesions are as follows: a) all of the above vulnerable plaque features, as well as: b) intralesional hemorrhage, c) intralesional thrombosis, d) endothelial erosion at lesion shoulders, e) neutrophil adhesion/presence in lesions, f) matrix metalloproteinase (MMP)>tissue inhibitor of metalloproteinase (TIMP) expression, and g) tissue factor expression.

We have not detected plaque fissure underneath luminal thrombus, which is a known feature associated with ACS in humans. We have, however, observed plaque erosion, which is a known alternative pathway to ACS.

Our analysis of the lesions further revealed the following:

a) There are nine or more metallaproteinases identified (matrix degrading proteins). To varying degrees, most have been associated with vulnerable plaques. From this panel, our data suggest that a key role can be assigned to MMP3 based on the prominence of its expression in the coronary artery lesions. Immunohistochemical analysis identifies MMP3 as a prominent player in vulnerable plaque evolution based on the following observations: 1) MMP3 is not detected in stable lesions; MMP3 is detected in unstable lesions; and 2) the ratio of MMP3 to TIMP3 expression is about 1:1 in early vulnerable plaque destabilization, but increases markedly in later stages of vulnerable plaque destabilization. The presence of MMP3, and the ratio of MMP3 to TIMP3 markers, thus can provide a detection paradigm for identification of vulnerable plaques in human patients.

b) Contrary to consensus views of vulnerable plaques, intralesional hemorrhage and thrombosis (hallmarks of unstable plaques underlying unstable angina) can occur in lesions with thick fibrous caps with significant numbers of smooth muscle cells. Thus, thrombosis does not occur only in the expected lesions with thin fibrous cap and a paucity of smooth muscle cells. One clinical implication of this observation affects detection strategies: clinicians need to address combinatorial features of interlesional hemorrhage and thrombosis, and not just surface thrombosis.

c) Lesion occlusion can occur through accelerated lesion growth through intralesional hemorrhage and/or thrombosis and/or foam cell enrichment. This unstable plaque phenotype (intralesional hemorrhage and/or thrombosis) likely underlies the failure of thrombolytic therapy in humans with acute coronary syndromes, since the thrombus is not on the surface exposed to thrombolytic therapy.

d) Because of identical localization, the features of the early coronary artery lesion that eventually develops into the vulnerable plaque can be investigated in our animal models through time course analysis. Identification of this early lesion phenotype is a very important target in cardiovascular research. With its identification, clinical imaging tools can be devised and measures can be developed to prevent evolution into unstable plaques.

e) The animal model demonstrates the importance of elevated triglycerides, increased VLDL, and low HDL as proatherogenic, even in the absence of an elevated LDL level, which is commonly thought one of the major risk factors for atherogenicity.

The transgenic model of the invention is unique in its development of the full spectrum of coronary artery disease which simulates human histopathology: stable lesions, vulnerable plaques, and plaque destabilization (plaque erosion, inflammation, foam cell enrichment, thrombus formation, intralesional hemorrhage, and resultant vessel occlusion). Furthermore, the phenotype of the animals of the invention results in decreased survival, thus simulating acute coronary syndrome (ACS) in humans. This ACS-phenotype is unique in that no other transgenic and/or non-transgenic animal model develops this end-stage phenotype in a predictable manner on regular chow (non-cholesterol enhanced as is necessary for primate and rabbit models) and with controlled genetic backgrounds (controlled genetic backgrounds are not currently available in primate and rabbit models). Other features and advantages of the invention will be apparent from the following detailed description, and from the claims.

### BRIEF DESCRIPTION OF THE DRAWINGS

FIGS. 1a-c show results of four transgenic Dahl S rat founder lines with the hCETP transgene.

(a) Schematic of the transgene: transgenic founders were produced containing the human cholesteryl ester transfer protein (hCETP) cDNA (1.57 kb XbaI/HindIII DNA fragment; SEQ ID NO: 1) regulated by the human Apolipoprotein CIII (ApoC3) promoter (1.435 kb) (SEQ ID NO: 2) and using the SV40 polyadenylation signal. Restriction enzymes: E, EcoRI; X, XhoI; H, HindIII; B, BamHI. The 3.56 kb transgene was linearized with EcoRI/BamHI.

(b) Southern blot analysis corroborated all founder lines and documented different integration sites and intact transgene based on DNA restriction fragments greater that the transgene size. The relative copy numbers are as follows: Tg53 (lane 1)>Tg25 (lane 2) >Tg22 (lane 3)=Tg21 (lane 4). Under stringent hybridization conditions, no CETP DNA fragments were detected in control Dahl S rat (lane 5).

(c) RNA blot analysis detected abundant transgene expression in the liver in Tg[hCETP]53 (lane d) and Tg[hCETP]25 (lane c) lines correlating with their CETP activity and lipid profiles (see below). Minimal message was detected in the liver in the low expressing lines (Tg[hCETP]21=lane a; Tg[hCETP]22=lane b) consistent with their CETP activity levels and lipid profiles. No CETP mRNA was detected in liver or small intestine of control non-transgenic Dahl S rat (lane e).

FIGS. 2A-C show comparative densitometric scans of Coomassie blue-stained nondenaturing gels of lipoproteins of density less than 1.063 g/ml. Plasma from 6 month old control non-transgenic (A), transgenic Tg25 (B) and Tg53 (C) Dahl S rats were analyzed revealing a predominant VLDL peak, and a very low LDL peak. Major peaks are indicated by their calculated particle diameters A in regions of the gel containing VLDL, IDL, and LDL.

FIGS. 3a-f show photomicrographs of the histological assessment of atherosclerotic lesion in aorta, coronary and intramyocardial arteries of transgenic Tg[hCETP]53 Dahl S rats compared with non-transgenic Dahl S rats at 6 months, on regular rat chow. At 6 months of age on regular rat chow, lesion were noted in the aorta, coronary and intramyocardial arteries of transgenic Tg[hCETP]53 Dahl S rats.

(a) Van Gieson elastic stain (original magnification 4×), showed a larger fibromuscular lesion with a huge necrotic core (orange in color, white arrow) on the inner curvature of the ascending aorta pointing towards the direction of flow; well encapsulated with fibromuscular tissue; prominent elastic laminae. Lesion occlusion of the ascending aorta is about 60%. Bar=250 microns.

(b) H&E staining (original magnification 20X) shows the right coronary artery(RCA) of Tg[hCETP]53 rat with an eccentric lesion. Inflammatory cells are noted in the adventitia (O). Similar lesions were noted in other Tg[hCETP]53 rats; foam cells, globular lipid deposits in both intima and media, but specific to the lesion side only; inflammatory cells in the adventitia, again only on the lesion side. Bar=50 microns.

(c) H&E staining (original magnification 40×) more distally, fibroatheroma resulting in 50% occlusion with large globular lipid deposits and fibrocellular cap. The internal elastic lamina ( [symbol] ) and the endothelium (Δ) are marked. The media adjacent to the lesion is marked by paucity of nuclei compared to media adjacent to the non-lesion areas. Bar-25 microns.

(d) H&E staining (original magnification 40×) left ventricular (LV) intramyocardial artery with thrombosis and almost complete occlusion of the lumen, and abnormal media and intimal disarray. In contrast, no atherosclerotic lesions were detected in the non-transgenic Dahl S controls—in the aorta as well as in the coronary arteries.

(e) RCA in anon-transgenic Dahl S rat heart (original magnification 20×) shows a normal tunica intima with no atherosclerotic lesions. The proximity of the internal elastic lamina ( [symbol] ) and endothelium (Δ) demonstrate the absence of any intimal thickening; (m), media.

(f) H&E (original magnification 40×) LV intramyocardial artery in the non-transgenic Dahl S control with no foam cells in the media, no intimal thickening or distortion, and no thrombosis.

FIGS. 4a-c show photomicrographs of the histological assessment of lesion site-specific activated endothelium-leukocyte adhesion and adventitial inflammatory response. H&E staining, original magnification 100×.

(a) high magnification detects monocyte (a few are indicated ⋄) adhesion to the endothelium (Δ). Adhesion is detected only at the lesion site in the eccentric coronary artery lesion shown in FIG. 4b. This lesion typified the lesions observed in several Tg[hCETP]53 Dahl S rats. Endothelial cells are distinct (A) from the monocyte/leukocyte (⋄). The RBCs within the lumen contrast the increased number of moncyte/leukocyte “rolling: on or attached to the endothelium of Tg[hCETP]53 rat. The lipid-laden thickened intima is demarcated between the internal elastic lamina ( [symbol] ) and the endothelium (Δ). Foam cell, globular lipid deposits, cholesterol crystals and increased cellularity are noted in the intima (i); the media (m) is noted.

(b) another region of the same artery shown in FIG. 4^{a}, cholesterol crystals and a few red blood cells are noted in the intima (▴); frayed internal elastic lamina ( [symbol] ). Intense inflammatory response is evident in the adventitia. No inflammatory response is detected in the non-lesion side (FIG. 4b). Decreased number of nuclei are noted in the media (m); foam cells are noted in the media.

c) a similar-sized coronary artery in non-transgenic age-matched Dahl S rat exhibits no intimal lesions with distinct endothelial cells (Δ) and internal elastic lamina ( [symbol] ). There is no leukocyte-adhesion to the endothelium; no lipid deposits or foam cells in the sub-endothelial, space or media (m); and no inflammatory response in the adventitia (ad) in control Dahl S rat artery.

FIGS. 5a-c show photomicrographs of detection of intramyocardial arterial thrombosis and myocardial infarction in Tg[hCETP53] rat hearts.

a) LV intramyocardial artery with occlusive thrombus (t). (PTAH, original magnification 40×.) Fibrin ( [symbol] ) in the thrombus stains blue-purple with PTAH. Bar=4 microns).

b) LV intramyocardial artery with an occlusive thrombus (t), (H&E, original magnification 100×). The endothelium and media are in disarray containing foam cells and lipid deposits. A protruding intimal lesion is noted (♦); foam cells are noted in the thickened intima, as well as in the media.

c) depicts an infarcted region with a relatively acellular fibrosed area (MI) in the left ventricle of a Tg[hCETP]53 rat heart with multiple thrombosed intramyocardial arteries. Bar=10 microns.

FIGS. 6a-f show photomicrographs of immunohistochemical analysis of serial sections of a representative coronary artery occlusive fibroartheromatous lesion.

a) intense vascular cell adhesion molecule-1 (VCAM-1) brown-horseradish peroxidase immunostaining ( [symbol] ) is detected in the endothelium (∇) and deep within the intimal lesion especially surrounding large globular areas presumably remnants of lipid deposits. VCAM-1 immunostaining is also detected in the media. VCAM-1 immunostaining is restricted to the lesion side. All nuclei are stained blue by Gill's hematoxylin stain.

b) high magnification corroborates VCAM-1 immunostaining ( [symbol] ) in foam cells flanking glocular deposits in the intima and media (m), as well as in the endothelium (∇).

c) ED1 positive immunostaining of the flanking serial section marks macrophages in the vicinity of intense VCAM-1 immunostaining FIG. 6a and b.

d) monocyte chemocattractant protein-1 (MCP-1) immunostaining ( [symbol] ) is detected in the endothelium (∇) overlying the lesion, as well as deep within said intimal lesion. A frayed internal elastic lamina is noted. Immunostaining is again specific to the lesion side.

e) CD3+ immunostaining is detected in a few cells within the intimal lesion marking T-cells.

f) negative serum control corroborates specificity of positive staining in panels 6a-e; nuclei are stained light blue-violet by Gill's hematoxylin stain. Original magnification:20× for 6a, 100× for 6b-f.

### DETAILED DESCRIPTION OF THE INVENTION

Here we demonstrate that on regular rat chow, Dahl salt-sensitive hypertensive (Dahl S) rats transgenic for hCETP, Tg[hCETP], exhibited hypertriglyceridemia, hypercholesterolemia, and decreased HDL levels—all worsening with age. Two high expresser lines exhibited coronary artery disease with relative severity proportional to lipoprotein abnormality. The highest expresser lines exhibited coronary artery disease with relative severity proportional to lipoprotein abnormality. The highest expresser transgenic line, Tg[hCETP]53, exhibited coronary artery disease and myocardial infarction at 6 months, and subsequent decreased survival compared with control non-transgenic Dahl S rats. Myocardial infarction and decreased survival has not been reported in Tg[CETP] mouse models. Our results show that CETP is proatherogenic and that the exacerbation of atherosclerosis by polygenic hypertension can be genetically modeled meeting Koch's hypothesis testing requirement.

These findings differ from various mouse atherosclerosis models demonstrating the necessity of complex disease modeling in different species. Our data demonstrates that triglyceride rich, very low density lipoprotein (VLDL) does contribute to atherosclerosis lesion development. The modeling of the interaction of polygenic hypertension and atherosclerosis in Tg[hCETP] Dahl S rats substantiates a longstanding epidemiological observation in humans.

Combined Hyperlipidemia in Tg[hCETP] Dahl S Hypertensive Rats

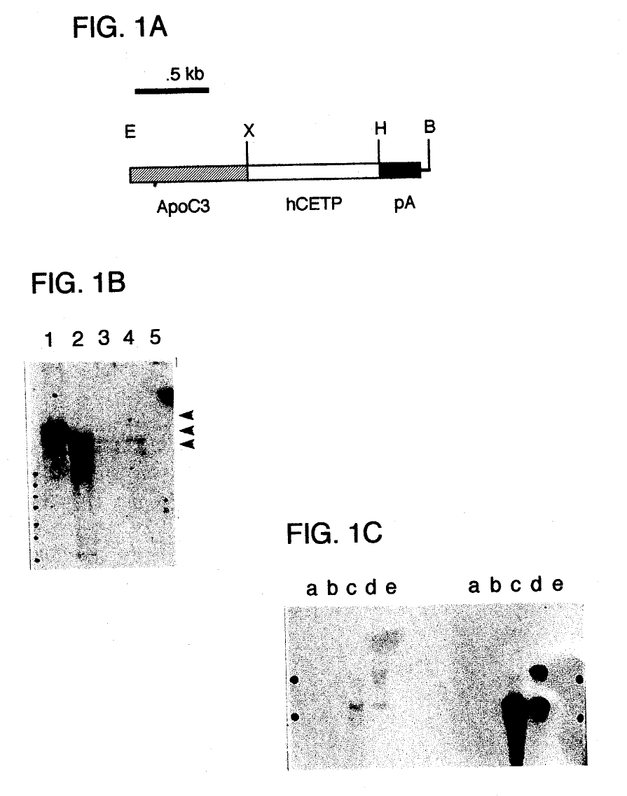

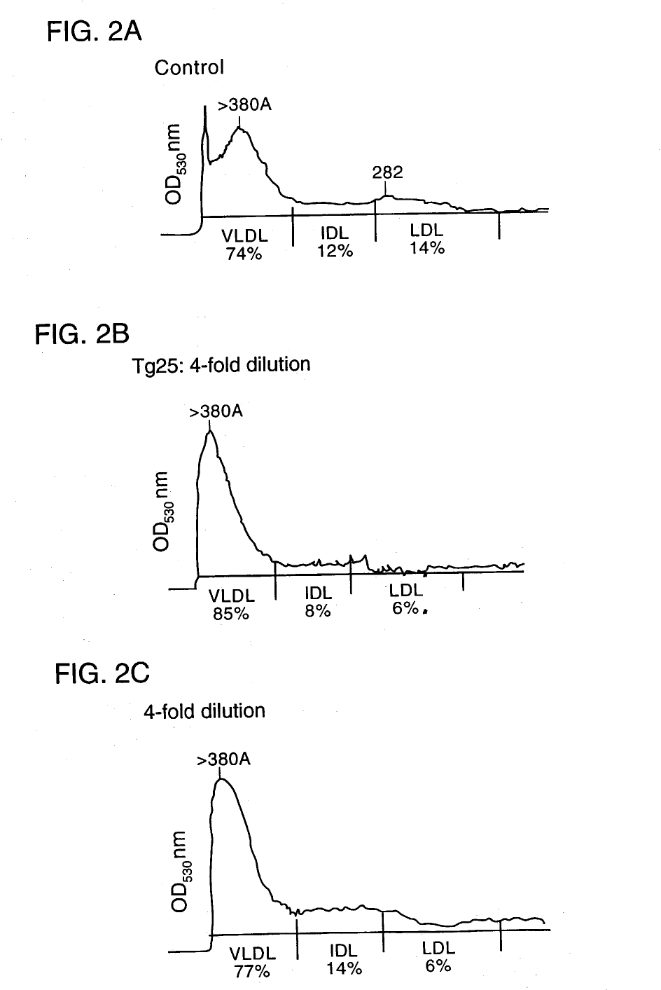

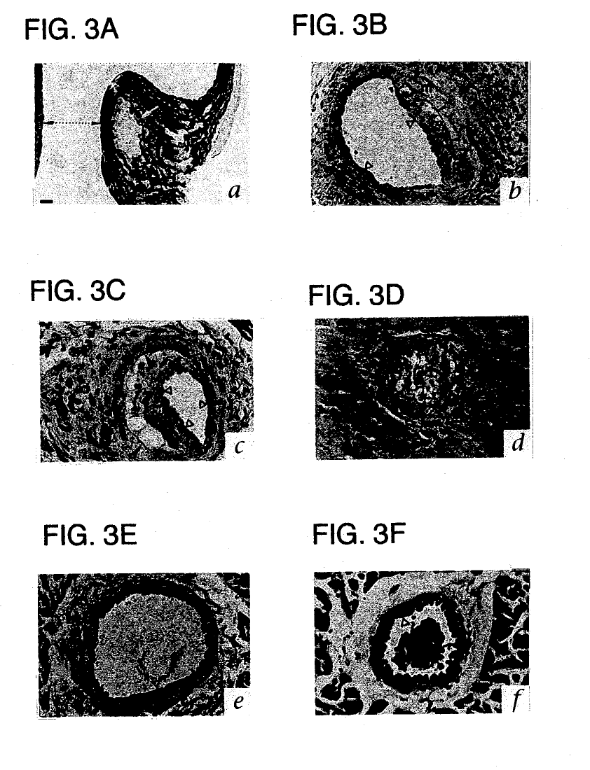

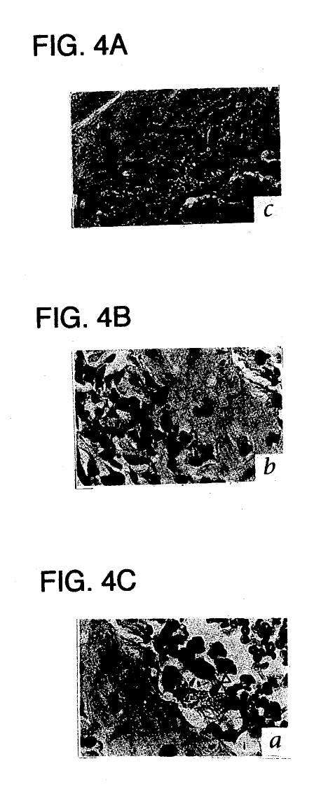

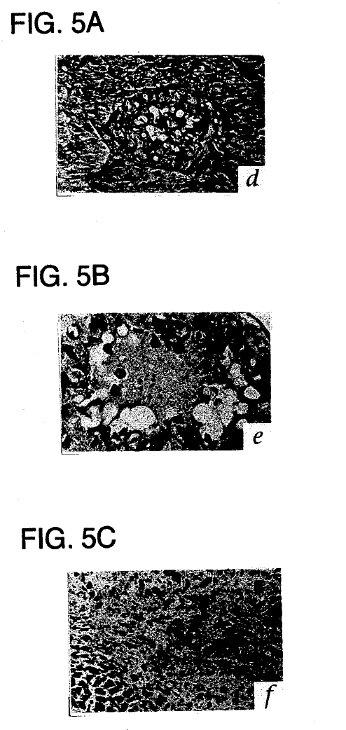

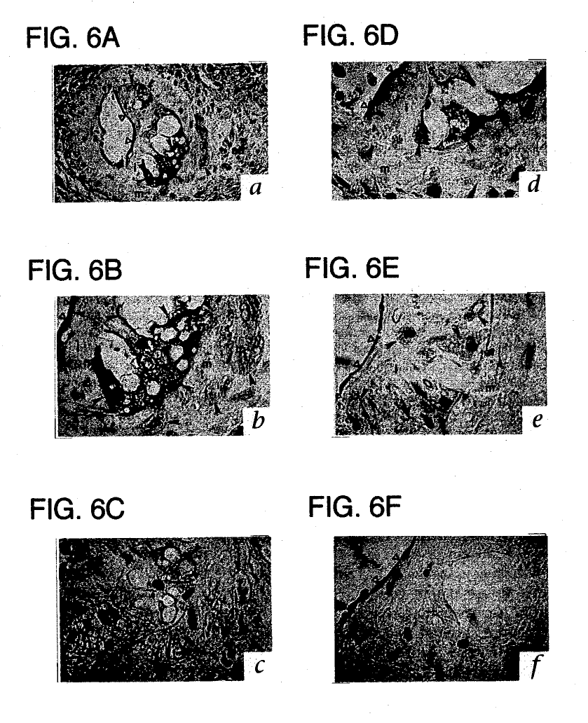

Using genetically hypertensive Dahl Salt-sensitive HSD (S) inbred rats (Harlan Sprague Dawley, Indianapolis, Ind.)¹⁷, four transgenic lines were developed in the Dahl S rat strain with the recombinant transgene comprised of the human Apolipoprotein CIII (ApoC3) promoter (−1114 to +24; GenBank Accession No. M60674; SEQ ID NO: 2), human cholesteryl ester transfer protein (hCETP) cDNA (GenBank Accession No. NM_{—}000078; SEQ ID NO: 1), and SV40 polyadenylation signal sequence (FIG. 1a). Southern blot analysis detected the transgene in all four transgenic lines arbitrarily numbered as Tg53 (lane 1), Tg25 (lane 2), Tg22 (lane 3), and Tg21 (lane 4) in contrast to its absence in control non-transgenic Dahl S rats (FIG. 1b). RNA analysis (FIG. 1c) detected minimal expression in Tg21 (lane a) and Tg22 (lane b), and marked expression in Tg25 (lane c) and Tg53 (lane d) lines, with intestinal expression (left panel) much less than hepatic expression. Transgene hCETP RNA levels correlated with relative transgene copy number (FIG. 1b) Tg53=Tg25>>>Tg21=Tg22 (FIG. 1d). The larger CETP transcript detected in Tg[hCETP]53 most likely represents varying lengths of 3′ un-translated region from different polyadenylation start sites. All four transgenic lines were germ-line integrated. All subsequent analyses were done on male hemizygous transgenic rats from each Tg[hCETP] line and compared with non-transgenic male littermates; all were maintained on regular rat chow.

At 6 months of age, CETP levels were determined and revealed two high expresser lines, Tg53>Tg25;, and two low expresser lines Tg22>21 (Table 1) consistent with hCETP transgene copy number and hepatic RNA levels. At 6 months of age, marked combined hyperlipidemia with significant elevation of both 24-hour fasting total cholesterol and triglyceride levels in both high expresser Tg[hCETP]25 and 53 rats in contrast to low expresser Tg21 and Tg22 rat lines, as well as littermate control non-Tg Dahl S rats. A dose-response CETP effect is apparent with greater CETP activity in Tg53 rats corresponding to greater total cholesterol and triglyceride levels, followed by Tg25>Tg22>Tg21 (Table 1). Consistent with the association of CETP with elevated TG/decreased HDLc ratio in humans, an inverse effect is seen of HDLc levels with markedly lower HDLc levels in high expresser lines Tg53<Tg25 compared with low expresser lines, Tg21 and Tg22, and controls. A dose response effect is also apparent with HDLc decrease following CETP levels inversely.

The distribution of subspecies of lipoproteins of density less than 1.063 g/ml was examined for both high expresser transgenic lines, Tg[hCETP]25 and 53, and compared with non-transgenic Dahl S rats at 6 months of age. Densitometric scans of Coomassie blue-stained non-denaturing 2-16% polyacrylamide gradient gels detected very low density lipoprotein (VLDL) as the major peak (calculated particle diameter>380 Å) in both Tg[hCETP]25 and 53 rats in contrast to control non-transgenic Dahl S rats (FIG. 2).

Atherosclerosis Lesion Phenotype

To determine the atherosclerosis lesion phenotype of this model, serial histopathological analysis was performed on hearts from 6 month-old Tg[hCETP] 53 (n=6), Tg[hCETP]25 (n=2), and control non-Tg (n=6) Dahl S rats; as well as post-mortem (>6 months) on homozygous Tg[hCETP]25^{=/=} (n=1) rat heart. In contrast to the epicardial coronary artery course in humans, both left and right coronary arteries emerged from the aortic root within the myocardium in the rat heart. A representative panel of photomicrographs (FIGS. 3, 4 and 5) shows lesions detected in this rat model. Multi-level arterial lesions were noted in the Tg[hCETP]53 line at 6 months of age on regular rat chow; aortic (FIG. 3a), coronary (FIG. 3b) and intramyocardial arterial (FIGS. 3c and 3d) lesions. The aortic lesion is characterized by a thick fibromuscular cap with prominent elastic laminae and necrotic core, that results in significant luminal occlusion (FIG. 3a). This aortic lesion was detected in only 1 out of 6 six month-old Tg[hCETP]53 rats analyzed. In contrast, lesions in the coronary arteries (FIG. 3b) were reproducibly detected in Tg[hCETP]53 rats (6/6 rats) and had similar characteristics in both right coronary artery (6/6 arteries examined) and left coronary artery (2/2 arteries examined), as well as in the Tg25^{+/+} rat heart examined at death. Distal to the lesion shown in FIG. 3b in the right coronary artery, an eccentric fibroatheromatous lesion is detected with 50% luminal occlusion, thickened intima with foam cells, globular lipid deposits, and an endothelialized fibromuscular cap (FIG. 3c). A representative lesion, observed in left ventricular intramyocardial arteries (FIG. 3d), shows lumen occlusion due to thrombosis with irregular intimal thickening and foam cells in the media. In contrast, serial analysis of multiple control non-transgenic Dahl S rats did not detect atherosclerotic lesions in the aorta, coronary (FIG. 3e) or intramyocardial arteries (FIG. 3f) at 6 months of age on regular rat chow. Lesions were not detected in low expresser Tg[hCETP]21 and 22 transgenic lines.

High magnification analysis of lesions revealed evidence of lesion-specific endothelial cell activation characterized by leukocyte adhesion consistent with atherosclerosis pathogenesis¹⁸. Leukocytes were detected adhered to the endothelial surface and in the subendothelial space of lipid-laden coronary artery lesions marked by intimal increase, globular lipid deposits, cholesterol clefts and foam cells (FIG. 4a). Leukocyte adhesion to the endothelium was specific to lesioned areas in contrast to non-lesioned areas within the same vessel cross section. Likewise, adventitial inflammatory response was noted adjacent to lesioned areas only (FIGS. 3b and 4b). Additionally, the media in lesioned areas exhibited foam cells, fewer nuclei and were paler on hematoxylin and eosin (H&E) staining in contrast to the media in the opposing non-lesion area of the same vessel section (FIGS. 3b, 4a,b). Specificity of findings in Tg[hCETP]53 rats was ascertained by the non-detection of said findings in the aortas and coronary arteries of non-transgenic Dahl S rats (FIGS. 3e, 3f, 4c).

High magnification analysis of intramyocardial resistance artery lesions detected foam cells in the media, irregular endothelium and varying degrees of luminal occlusion by thrombi (FIGS. 3d, 5a and 5b). Staining of serial sections with thrombi indicated fibrin by positive staining with Mallory's phosphotungstic acid hematoxylin stain (PTAH). Entrapped red blood cells were detected in the thrombi marking residual patency (FIGS. 5a and 5b). Histological changes consistent with myocardial infarction were detected in the left ventricle in Tg[hCETP]53 Dahl S rat hearts characterized by focal reorganization and spotty neovascularization (FIG. 5c), as well as by focal cardiomyocyte coagulative necrosis and inflammatory infiltrates. Evidence for thrombosis, myocardial infarction or decreased survival has not been described in the ApoE null mutant mouse model despite prominent atherosclerotic lesions¹⁶.

To further investigate molecular events underlying late-occurring lesion-specific leukocyte adhesion (FIG. 4a) which is typically implicated in lesion initiation immunohistochemical analysis of serial sections flanking the eccentric occlusive lesion depicted in FIG. 3c was done. We investigated two key players in endothelial activation implicated in atherosclerosis lesion initiation: vascular cell adhesion molecule-1 (VCAM-1)¹⁹ and monocyte chemoattractant protein-1 (MCP-1)²⁰. As shown in FIG. 6, intense lesion-specific VCAM-1 immunoreactivity was detected in luminal endothelium, within the lesion in sub-endothelial intimal lipid-laden cells concentrated in proximity to globular lipid deposits (FIG. 6a, b), and in distinct cell groups in the media adjacent to the lesion. The flanking serial section detected ED1+ macrophages in proximity to intense VCAM-1 staining, with some containing intracellular lipid (FIG. 6c). MCP-1 immunoreactivity was detected in luminal endothelium and in cells surrounding globular lipid deposits (FIG. 6d). The area of MCP-1 expression was less than VCAM-1. A few CD3+ T-cells were detected within the occlusive intimal lesion (FIG. 6e). Specificity of immunoreactivity was ascertained by negative controls run in parallel (FIG. 6f), as well as by the distinctly lesion-restricted sites of immunoreactivity. The detection of leukocyte adhesion and intense VCAM-1 and MCP-1 staining in lesions well beyond initiation at 6 months of age, which is close to the mean age at early death of Tg[hCETP]53 rats, suggests that endothelial activation in this model is not limited to lesion initiation, but may also be involved in accelerated lesion progression.

Impact of Atherosclerosis Phenotype on Survival

In order to assess significance of the atherosclerosis phenotype in Tg[hCETP] rats, we determined life span on regular rat chow (Table 1). Decreased survival was detected in Tg[hCETP]53 Dahl S male rats in contrast to non-transgenic Dahl S male rats (t-test P<0.03). Decreased survival was not detected in the low expresser transgenic lines: Tg21 and Tg22. Survival in Tg[hCETP]25 rats decreased slightly but did not reach significance. Decreased survival has not been reported in atherosclerosis mouse models^{16,21}.

To clarify the most likely cause of death, blood pressure measurements were obtained by radiotelemetric 24-hour non-stress measurements. Analysis at 5 months of age on regular rat chow revealed that Tg[hCETP]53 rats (SBP=166±1.8 mmHg, n=7) exhibit less hypertension than age-matched Dahl S non-transgenic controls (SBP=186±4.1 mmHg, n=11), ANOVA P=0.001. These data eliminate hypertension as the sole cause of decreased survival thus pointing to the observed coronary heart disease as the most likely cause of shortened lifespan.

CETP is a Determinant of Combined Hyperlipidemia and Decreased HDLc

The lipid profiles in Tg[hCETP]25 and 53 rats differ from transgenic CETP mouse models that exhibited an isolated decrease in HDL cholesterol and did not exhibit hypertriglyceridemia regardless of transgene promoter—metallothionein²², cognate hCETP 5′ flanking region, or ApoA1 gene promoter²³, and regardless of level of expression²². Phenotype differences in lipoprotein profiles can be due to one or a combination of the following factors: higher transgene hCETP expression in Tg[hCETP]25 and 53 rat lines compared with murine models; species-specific factors that allow human CETP to interact more efficiently with endogenous components of rat lipid metabolic pathways compared with mouse concordant with observations in bigenic [hApoA1×hCETP] mouse model²⁴ and/or the presence of putative positive modifier genes in the Dahl S rat strain.

The observed high triglyceride-low HDLc phenotype in Tg[hCETP]53 and 25 Dahl S rats parallels observations in humans demonstrating that CETP activity contributes to a high-triglyceride, low-HDL phenotype²⁵. The hypertriglyceridemia cannot be attributed to insulin resistance reported in Dahl S rats²⁶, since all transgenic lines and controls are from the inbred Dahl S rat strain. With insulin resistance a commonality, hypertriglyceridemia should have been a common trait but it is not. Control non-transgenic and low expresser Tg[hCETP]21 and 22 Dahl S rats do not exhibit hypertriglyceridemia on regular rat chow (4% fat, 0.02% cholesterol) or on an atherogenic high fat, high cholesterol (HFHC: 15% fat, 1% cholesterol, 0.5% sodium cholate) diet²⁷ in marked contrast to Tg[hCETP]53 and 25 rat lines. Moreover, the dose response effect of CETP activity on triglyceride levels in Tg53 and Tg25 rats argues positively for a transgenic CETP-derived effect over other putative factors. These data define CETP as a molecular determinant of combined hyperlipidemia and decreased HDLc in the Dahl S rat genetic background.

Differential Atherosclerosis Phenotype in Transgenic CETP Rat Models

The atherosclerosis phenotype exhibited by Tg[hCETP] rats contrasts the phenotype observed in Tg[CETP] mouse models given the identical transgene product. The differences are not pathogenic subtleties. The differential highlights are: 1) spontaneous atherosclerosis in Tg [hCETP] rats contrasts the dietary-induced atherosclerosis in Tg [primate CETP]¹³ and Tg[hCETP]¹⁵ mice; 2) spontaneous combined hyperlipidemia, low HDLc in Tg[hCETP] rats contrasts the isolated decrease in HDLc and minimal hypercholesterolemia in Tg[CETP] mice^{15,22}; 3) predominance of atherosclerotic lesions in the rat coronary artery system contrasts the predominance of aortic root lesions in Tg[CETP] mice^{13,22}; 3) predominance of atherosclerotic lesions in the rat coronary artery system contrasts the predominance of aortic root lesions in Tg[CETP]mice^{13,22}; and most significantly, 4) myocardial infarction and decreased survival in Tg [CETP] rats contrasts the absence in all reported transgenic mouse models reported to date—inclusive of the prototype ApoE null model with similar hypercholesterolemia^{16,21,28-29}, and the hybrid [TgApoC3×ApoE-/-] mouse model with similar levels of hypercholesterolemia and hypertriglyceridemia³⁰. These data reiterate that modeling human diseases requires investigation in multiple model systems and demonstrate the limitations of atherosclerosis mouse models²¹.

With respect to the atherosclerosis lesion phenotype, the a priori significant mouse-rat differences that contribute to the differential lesion phenotype likely involve several factors. Foremost is the presence of significant hypertension, albeit reduced when compared with control non-transgenic Dahl S rats. Epidemiological observations in humans have demonstrated that hypertension accelerates but does not cause atherosclerosis⁴. Non-hypertension related other-predisposing genetic factors in the Dahl S rat background cannot be ruled out/in at present, however, we note that the Dahl S strain is athero-resistant since it does not develop any atherosclerotic lesions on an atherogenic HFHC diet²⁷. This contrasts with the C57/B6 mouse strain which is athero-susceptible—atherosclerotic lesions develop on the identical atherogenic diet—and which is the background stain of transgenic CETP mouse models. Secondly, the significantly higher level of activity of the complement system in rats compared with mice³² might be a key determinant of the observed robust atherosclerosis phenotype, since complement activation has been implicated as a link between lipoprotein deposition and subsequent lesion development in human atherosclerosis³³. Further studies are necessary to dissect genetic modifiers, role of complement activation, and putative role(s) of discrete hypertension gene(s) in the atherosclerosis-hypertension interaction, however a strategic model is at hand.

The significance of rat-to-mouse differences in the study of atherosclerosis mechanisms is marked given that although increased susceptibility to dietary atherosclerosis was observed in non-hypertriglyceridemic simian and human CETP transgenic mice compared to control^{13,15}, inhibition, and not induction, of early development of early atherosclerotic lesions was observed in bigenic and trigenic hypertriglyceridemic hCETP mice—Tg[hApoC3×hCETP] and Tg[hApoA1×hApoC3×hCETP] mice¹⁵. Again, this is counterintuitive to human epidemiological observations where cumulative evidence implicate hypertriglyceridemia as proatherogenic³⁴ and which this transgenic rat model supports. As with differential lipid profile phenotype, differential lesion phenotype between rat and mouse models reiterate the need for modeling complex human diseases in multiple model systems, as well as demonstrates complexity of the role of CETP requiring much study. Complexity of CETP role is highlighted once more with a recent study demonstrating that hCETP transgene expression increased mean lesion area 1.4 to 1.8-fold in ApoE- and LDL receptor null mutant mice¹⁴ supporting proatherogenicity of CETP, however no effects on myocardial infarction or survival were reported.

In contrast, the definitive robust phenotype in single transgenic Tg[hCETP]53 Dahl S rats clarifies several issues. This model demonstrates the following: that both increased CETP activity is proatherogenic; that elevated triglyceride-rich VLDL levels, in the absence of elevated low density lipoprotein, is a determinant of significant coronary artery atherosclerosis lesions; and that focal factors of atherogenesis dominate lesion development despite the presence of generalized consequences of both hypercholesterolemia and hypertension on endothelium, media and adventitia. Additionally, this model shows that severe combined hyperlipidemia decreases blood pressure levels in polygenic hypertension in male Tg[hCETP]53 and 25 rats to a certain extent, but that lesion development continues nevertheless—alerting one to a potentially misleading clinical scenario.

Cross Talk Paradigm of Hypertension-Atherosclerosis Interaction

The mechanistic analysis of the exacerbation of atherosclerosis by hypertension requires a strategic animal model which the Tg[hCETP]53 rat line provides. Although undoubtedly complex and realizing the limitations of animal models for human disease, paradigms of pathogenesis can be dissected which would have relevance to human disease. Given that hypercholesterolemia further impairs hypertension-induced endothelial dysfunction in Dahl S rats²⁷ and that both VCAM-1 and MCP-1 expression are increased by both hypertension³⁵⁻³⁷ and hypercholesterolemia¹⁹⁻²⁰, our observations of lesion-specific leukocyte adhesion and increased VCAM-1 and MCP-1 expression beyond the early initiation stages of atherosclerotic lesion development suggest that a putative hallmark of the exacerbation of atherosclerosis by hypertension is extended, if not persisting, endothelial activation. Since VCAM-1 is a potent adhesion molecule in atherosclerosis³⁸ preceding lesion formation¹⁹ and MCP-1 is an inducer of procoagulant tissue factor in atherosclerosis³⁹, we hypothesize that persisting endothelial activation through a resultant pro-inflammatory and prothrombotic cross talk paradigm underlies the exacerbation of atherosclerosis by hypertension. This paradigm would be consistent with current hypotheses on acute coronary syndrome in humans^{16,40,41} The identification of VCAM-1 and MCP-1 as two putative key players in this cross talk paradigm marks them as potential intervention targets. Not insignificantly, pro-inflammatory pathways as mechanisms for acceleration of atherosclerosis lesion progression draw parallels from post-transplantation accelerated atherosclerosis that is initiated by immunopathogenic pathways^{42,43}. Lesion development differs however, in that diffuse concentric proliferative fibrocellular intimal thickening typical of post-transplantation arteriosclerosis⁴⁴ are not detected.

The concurrent involvement of endothelium, media, and adventitia specific to eccentric coronary artery lesions suggest the hypothesis that hypertensive vascular pathology is permissive to atherosclerosis lesion progression, but that the trigger for lesion initiation remains dependent on atherosclerosis-specific pathways. The significant involvement of major and smaller cardiac vessels suggests the hypothesis that the effects of hypertensive vascular pathology on atherogenic pathway affect the whole coronary system in the Tg[hCETP] rat model.

Altogether, the significant coronary heart disease phenotype marked by concurrent multivascular layer and multi-sized vessel involvement in this first transgenic-atherosclerosis polygenic hypertension rat model suggest a cross-talk paradigm of converging common events underlies hypertension-atherosclerosis interaction predisposing towards accelerated lesion development and destabilization.

Lesion analysis of Tg53 rats at end stage revealed a spectrum of lesion phenotype in the same coronary artery consistent with observations in humans. More distal lesions simulated stable plaque phenotype. More proximal lesions simulated vulnerable plaque features: decreased smooth muscle cells, lipid-enriched area>>fibrous cap, thin fibrous caps with proximity of lipid area to lumen, extensive foam cell area. End-stage lesions depicted a framework of plaque destabilization with Type VI lesions with luminal thrombosis surrounded by areas of myocardial infarction, as well destabilized vulnerable plaques with increased foam cell area, endothelial erosion, intra-lesion thrombosis and hemorrhage resulting in lumen encroachment. End stage lesions exhibit intense matrix degrading MMP-3 expression as well as procoagulant tissue factor expression—simulating observations in human CAD lesions. The more severe CAD phenotype in Tg53 rats compared with moderate CAD phenotype in Tg25 rats is associated with differential lipid profiles marked by earlier onset of abnormalities in Tg53 and greater elevations of LDLtg and HDLtg at 10 weeks and 6 months of age in Tg53 rats compared with Tg25 rats.

Based on a reconstruction of post-mortem analysis of coronary lesions and their association with end-stage cardiac events, the concept of vulnerable plaque-destabilization as the “culprit plaque” in acute coronary syndromes was deduced. We investigated whether this would be simulated in the Tg53 rats that exhibit an atherogenic lipid profile, predominant CAD lesions at 6 months and decreased survival. Investigation at end-stage was carried out in Tg53 rats maintained on regular rat chow and housed in pathogen-free conditions, and limited to rats identified in distress and subsequently euthanized to obtain end-stage but ante-mortem heart specimens. Some Tg53 rats die suddenly, and some are detected in distress—the latter end stage group rats are analyzed here thus eliminating post-mortem histopathology changes as confounders.

Analysis of end-stage Tg53 rats exhibiting acute distress (n=5/5 rats) revealed a lesion phenotype consistent with the unstable plaque phenotype—destabilized vulnerable plaque—reconstructed from post-mortem human plaque analysis. Lesions exhibited cellular features consistent with unstable plaques: thin fibrous cap, lipid core>40% of lesion area, paucity of smooth muscle cells in the fibrous cap, foam cell rich, inflammatory changes such as leukocyte adhesion/infiltration. Distinct features of plaque destabilization associated with unstable angina are also noted such as intramural thrombi and intramural hemorrhage. A recent evolving association of neutrophils and unstable plaques was observed in tg53 rat end-stage lesions. Neutrophil adhesion and infiltration were observed in the aforementioned lesion displaying plaque destabilization features. Most significantly, plaque erosion at the shoulder region was noted with leukocyte adhesion in the vicinity, as well as intramural thrombosis and hemorrhage.

Further support for plaque destabilization occurring in Tg53 rat CAD lesions was obtained by immunohistochemical analysis. Consistent with unstable plaque features described in human CAD, immunohistochemical staining revealed expression of proteins linked to unstable plaques in humans with unstable angina, such as ongoing inflammation hallmarks, e.g., proinflammatory TNF-α, procoagulant tissue factor, and matrix degrading metalloproteinases, MMP-3. The fact that serial sections reveal that all three are present contemporaneously suggests a complex of plaque destabilization events with inflammation, matrix degradation, and procoagulant, contributing to a dynamic end-stage lesion phenotype.

Significance of the observed complex of atherosclerosis plaque destabilization phenotype was deduced from the observation that lesions with intramural thrombi exhibit severe occlusion, if not complete occlusion, associated with different stages of post-myocardial infarction histopathologicall changes such as inflammation, fibrosis, and loss of myocardiocytes.

Additionally, an overall framework of dynamic lesion destabilization was evident as different destabilization—features were noted in the same rat heart, along with stable plaque morphology. Interestingly, severely or completely occlusive lesions do not necessarily have luminal thrombi, suggesting that luminal thrombosis is not the only pathway for acute lesion occlusion. The data suggest the hypothesis that lumen occlusion can be attained by increased lesion mass through increased foam cell formation from plaque erosion at the shoulder region as well as from significant intramural thrombi.

Pinpointing the Earlier Lesion Phenotype Predisposed to Later Plaque Destabilization

Given the inbred strain of the transgenic rat lines and maintenance of identical experimental conditions for transgenic rats in analysis, it is valid to reconstruct lesion development analyzed by histopathology from a series of transgenic rat hearts analyzed at early and end-stage time points. Although rat-to-rat variability could be expected as pathophysiology is never absolute, trend analysis of multiple transgenic rats provides a reasonable confidence interval. Meeting these requirements, a framework of analysis based on the simple assumption that lesions at identical location reflect a priori a time course of lesion development becomes valid.

Our analysis revealed that the non-occlusive eccentric lesion at 6 months of age that exhibits endothelial activation marked by leukocyte adhesion is the one that develops into the vulnerable plaque that proceeds to plaque destabilization and occlusion through intramural thrombosis and hemorrhage, intramural foam cell accumulation and/or luminal thrombosis.

The more occlusive fibroproliferative lesion has been observed to progress to severe occlusion in the tg25 transgenic line but without signs of inflammation, enrichment of macrophages, intramural or luminal thrombi. Histochemical analysis reveals that these stable lesions do not exhibit much, if any, matrix metalloproteinase-3 staining, nor tissue factor staining in contrast to the intense immunostaining seen in destabilized vulnerable plaques. However, unstable plaques are observed at end-stage in Tg25 rats—but at a decreased percentage (6/8 tg25 male rats) in contrast to Tg53 (7/7 male rats). This differential end-stage lesion phenotype is associated with relatively longer survival in Tg25 rats compared with Tg53 rats.

Correlates of Differential Plaque Pphenotype Development A key question that facilitates translation to bedside-relevant issues is the investigation of the critical clinically accessible correlate of a predisposition to the vulnerable plaque phenotype. Because of the differential phenotype between Tg53 and Tg25, analysis of differential lipoprotein profiles would be insightful into determining whether there might be a putative key determinant of plaque vulnerability and/or destabilization. Observations in human CAD indicate that there has not been one clearcut determinant of plaque vulnerability. Lipoprotein profile analysis was done comparing Tg53, Tg25 and non-transgenic Dahl S rats at 10 weeks, 4 months and 6 months of age. Ultracentrifugation was done on fresh plasma to measure cholesterol and triglyceride levels in each lipoprotein class fraction. ApoB levels were also determined by immunoturbidemetric methods. Analysis revealed significant differences between non-trangsenic Dahl S rats and transgenic Tg53 and Tg25 in total cholesterol (TC), total triglyceride (TG) and cholesterol and triglyceride levels in all lipoprotein classes isolated by ultracentrifugation—VLDL, IDL, LDL and HDL (Table 2). The data demonstrate that increased human CETP expression in the Dahl S rat strain results in age-related increase in total plasma cholesterol, predominantly in the VLDL fraction while significantly lowering HDL, and age-related increase in total plasma triglyceride levels predominantly in the VLDL fraction but also significantly in the LDL and HDL fractions. LDLc is actually significantly decreased in both Tg25 and Tg53 rat lines compared with non-transgenic Dahl S rats at both 10 weeks and 6 months.

With the differential plaque phenotype exhibited in Tg53 rats compared with Tg25 rats resulting in decreased survival in Tg53 rats but not in Tg25 rats compared to control non-transgenic Dahl S rats, we investigated which lipoprotein profile would correlate with this differential CAD phenotype. Comparative analysis at 10 weeks and 6 months of age revealed that: 1) Tg53 rats exhibit worse lipoprotein profile abnormalities at 10 weeks of age compared with Tg25 rats but that at 4 months and 6 months of age, the lipoprotein profiles between Tg53 and Tg25 are more similar; 2) that it is LDLtg that is significantly increased at both 10 weeks and 6 months of age in Tg53 compared with Tg25 suggesting that this might be a critical determinant of the vulnerable plaque phenotype exhibited by Tg53 rats which results in a more severe CAD phenotype marked by decreased survival and myocardial infarction. Comparing the lesions observed in 16 month old ApoE null mutant, it is significant to note the paucity of cholesterol clefts in Tg53 rats and instead the predominance of large lipid globular remnants or large areas of foam cells. We hypothesize that by association of this lesion phenotype with differential lipoprotein profile, that the increased triglyceride levels and low HDLc might underlie this vulnerable lipid content in CAD lesions. The data also suggest that in addition to LDLtg, earlier onset, hence duration of lipoprotein abnormalities, in Tg53 rats most likely play a significant role in the development of a worse CAD phenotype in Tg53 rats compared with Tg25 rats.

Lesion Development and Progression in Perspective

Having controlled genetic background, environment and diet—an impossibility in humans yet a prerequisite to understanding of mechanisms, lesion analysis provides new insight as well solidifies previous notions:

different lesion stage and type contemporaneously exist in the same heart—dynamic process hence clinical detection of one type does not preclude the others;

reproducibility of lesion type-location association—reiterates mechanical shear stress/strain on vessel as initiating event;

vulnerable plaques in the proximal vessel; stable lesions more distally;

striking focality of vessel wall changes;

involvement of media and adventitia.

Within-lesion thrombosis and expansion suggests that lesion surface-targeted interventions such as current anti-thrombotic agents may not always work-lesion expansion via foam cell expansion perhaps underlies failed antithrombotic therapies—no matter how early.

### Methods

Generation of Transgenic Animals

Transgenic constructs are usually introduced into cells by microinjection (Ogata et al., U.S. Pat. No. 4,873,292). A microinjected embryo is then transferred to an appropriate female resulting in the birth of a transgenic or chimeric animal, depending upon the stage of development of the embryo when the transgene integrated. Chimeric animals can be bred to form true germline transgenic animals.

In some methods of transgenesis, transgenes are introduced into the pronuclei of fertilized oocytes. For some animals, such as mice, fertilization is performed in vivo and fertilized ova are surgically removed. In other animals, the ova can be removed from live, or from newly-dead (e.g., slaughterhouse) animals and fertilized in vitro.

Alternatively, transgenes can be introduced into embryonic stem cells (ES cells). Transgenes can be introduced into such cells by electroporation, microinjection, or any other techniques used for the transfection of cells which are known to the skilled artisan. Transformed cells are combined with blastocysts from the animal from which they originate. The transformed cells colonize the embryo, and in some embryos these cells form the germline of the resulting chimeric animal (Jaenisch, R., Science 240: 1468-1474, 1988).

ES cells containing an hCETP transgene may also be used as a source of nuclei for transplantation into an enucleated fertilized oocyte, thus giving rise to a transgenic animal. More generally, any diploid cell derived from embryonic, fetal, or adult tissue and containing an hCETP transgene may be introduced into an enucleated unfertilized egg. The cloned embryo is implanted and gestated within an appropriate female, thus resulting in a fully transgenic animal (Wilmut et al., Nature 385:810-813, 1997).

In general, expression of any transgene depends upon its integration position and copy number. After a transgenic animal having the appropriate transgene expression level and tissue-specific transgene expression pattern is obtained by traditional methods (e.g., pronuclear injection or generation of chimeric embryos), the animal is bred in order to obtain progeny having the same transgene expression level and pattern. There are several limitations to this approach. First, transmission of the transgene to offspring does not occur in transgenic chimeras lacking transgenic germ cells. Second, because a heterozygous transgenic founder is bred with a non-transgenic animal, only half of the progeny will be transgenic. Third, the number of transgenic progeny is further limited by the length of the gestation period and number of offspring per pregnancy. In view of these limitations, nuclear transfer technology provides the advantage of allowing, within a relatively short time period, the generation of many female transgenic animals that are genetically identical.

After the candidate transgenic animals are generated, they must be screened in order to detect animals whose cells contain and express the transgene. The presence of a transgene in animal tissues is typically detected by Southern blot analysis or by employing PCR-amplification of DNA from candidate transgenic animals (see, e.g., Ausubel et al., Current Protocols in Molecular Biology, John Wiley & Sons, New York, N.Y., 1998; see also Lubon et al., U.S. Pat. No. 5,831,141).

Transgene Design and Development

The full length XbaI/HindIII 1.57 kb hCETP cDNA (licensed from Dr. Alan Tall, Columbia University, New York, N.Y.; SEQ ID NO: 1) was directionally subcloned into pSV-SPORT1 upstream to the SV40 small t splice and polyadenylation signal sequence (Gibco/BRL Life Technologies, Grand Island, N.Y.). The EcoRI/XbaI 1.43 kb genomic fragment of the human ApoC3 promoter (−1411 to +24; SEQ ID NO: 2; generous gift from Dr. Vassilis Zannis, Boston University School of Medicine, Boston, Mass.) was then directionally subcloned upstream of the hCETP using EcoRI and XbaI restriction digest sites. The ApoC3 promoter was selected to stimulate the hepatic and intestinal expression patter of hCETP^{45,46}. Functionality of the ApoC3-hCETP transgene was previously validated in Chinese hamster ovary cells⁴⁷. The 3.56 kb transgene was excised with EcoRI/BamHI, purified and microinjected into Dahl S rat one-celled embryos as described¹⁷. Founders and transgenic offspring were identified by slot blot analysis and corroborated by Southern blot analysis as described¹⁷. Transgene expression was determined by RNA blot analysis of liver and intestine RNA from transgenic and non-transgenic age-matched rats as described¹⁷. Transgenic lines were maintained and bred on regular rodent chow (0.02% cholesterol, 4% fat).

Lipid Profile Analysis

Plasma samples were obtained after 24-fast in 1 mM EDTA. Plasma samples from the high expressers were markedly lipemic. Total cholesterol was measured using the Cholesterol CII kit based on an enzymatic colorimetric method (COD-PAP) (Wako Chemicals, Inc., Richmond, Va.). Total triglyceride was measured using the Triglyceride EGPO-DAOS method (Wako Chemicals, Inc., Richmond, Va.) according to manufacturer's instructions. HDLc was measured using an HDL-cholesterol kit based on an enzymatic colorimetric method after precipitation and removal of—lipoproteins (Wako Chemicals, Inc. Richmond, Va.). CETP activity was measured using the CETP Diagnescent Kit (Diagnescent Technologies, Inc., Bronxville, N.Y.) per manufacturer's specifications. All samples were tested in duplicate.

Nondenaturing polyacrylamide gradient gel electrophoresis of lipoproteins in the density less than 1.063 g/ml ultracentrifugal reaction was carried out using 2-16% polyacrylamide gradient gels (Pharmacia, Piscataway, N.J.) stained for protein with Coomassie brilliant blue R-250 and subsequent determination of diameters of major bands calculated from densitometric scans using calibration standards as described⁴⁸.

Histological Analysis

At six months of age, transgenic and control non-transgenic rats were euthanized and tissues collected after blood was obtained for analysis. The heart was carefully removed with the aortic arch attached, washed in cold phosphate buffered saline, and then preserved in 4% PBS-buffered paraformaldehyde. Serial frontal sections of the paraffin-embedded heart and aortic arch were cut and stained with hematoxylin and eosin (H&E), Masson Trichrome and Van Gieson elastic stain (HistoTechniques, Powell, Ohio). Slides with thrombi in intramyocardial arteries were also stained with Mallory's phosphotungstic acid hematoxylin (PTAH) to detect fibrin. Slides were analyzed by light microscopy. Photomicroscopy was done on a Nikon Optiphot microscope.

Immunohistochemistry

Immunohistochemical analysis was done on serial section flanking section with lesions identified by H&E staining. Antigen-unmasking of 4%-PBS fixed, paraffin-embedded sections was performed by heating twice in 0.01 M sodium phosphate, pH 6.8 at 95°Cx5, minutes. Antibodies, control sera and horse-radish peroxidase staining kits were obtained from Sta. Cruz Biotechnology Inc., Sta. Cruz, Calif.) and used following manufacturer's specifications. Antibodies were used at 1:50 dilutions; primary antibody was applied overnight at 4° C.

Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth, and follows in the scope of the appended claims.

### REFERENCES

1. MRFIT Multiple Risk Factor Intervention Trial Research Group. Relationship between baseline risk factors and coronary heart disease and total mortality in the Multiple Risk Factor Intervention Trial. Prev. Medicine 15, 254-273 (1986).

2. Borhani N. O. Epidemiology of risk factors for cardiovascular disease. In, Atherosclerosis Beyond Cholesterol 3-16 (Audio Visual Medical Marketing, Inc., New York, N.Y. 1992).

3. Chobanian A. V. & Alexander R. W. Exacerbation of atherosclerosis by hypertension: potential mechanisms and clinical implications. Archives of Internal Med 156, 1952-1956 (1996).

4. Chobanian A. V. 1989 Corcoran Lecture: adaptive and maladaptive responses of the arterial wall to hypertension. Hypertension 15, 666-674 (1990).

5. Chobanian A. V. et al. Influence of hypertension on aortic atherosclerosis in the Watanabe rabbit. Hypertension 14, 203-209 (1989).

6. Brasen J. H., Harsch M., & Niendorf A. Survival and cardiovascular pathology of heterozygous Watanabe heritable hyperlipidemic rabbits treated with pravastatin and probucol on a low-cholesterol (0.03%)—enriched diet. Virchows Arcn 432, (6):557-562 (1998).

7. Shiomi M., Ito T., Shiraishi M., & Watanabe Y. Inheritability of atherosclerosis and the role of lipoproteins as risk factors in the development of atherosclerosis in WHHL rabbits: risk factors related to coronary atherosclerosis are different from those related to aortic atherosclerosis. Atherosclerosis 96 (1), 43-52 (1992).

8. Guyard-Dangeremont V., Desrumaux C., Gambert P., Lallemant C., & Lagrost L. Phosopholipid and cholesteryl ester transfer activities in plasma from 14 vertebrate species. Relation to atherogenesis susceptibility. Comp Biochem Physiol B Biochem Mol Biol 120, 517-525 (1998).

9. Moulin P. Cholesteryl ester transfer protein: an enigmatic protein. Hormone Research 45, 238-244 (1996).

10. Inazu A. et al. Increased high-density lipoprotein levels caused by a common cholesteryl-ester transfer protein gene mutation. New England J Med 323, 1234-1238 (1990).

11. Yamashita S. et al. Characterization of plasma lipoproteins in patients heterozygous for human plasma cholesteryl ester transfer protein (CETP) deficiency: plasma CETP regulates high-denisty lipoprotein concentrations and composition. Metabolism 40, 756-763 (1991).

12. Zhong S. B., Sharp D.S., Grove J. S., Bruce C., Yano K., Curb J. D., & Tall A. R. Increased coronary heart disease in Japanese-American men with mutation in the cholesteryl ester transfer protein gene despite increased HDL levels. J Clin Invest 97, 2917-2923 (1996).

13. Marotti K. R. et al. Severe atherosclerosis in transgenic mice expressing simian cholesteryl ester transfer protein. Nature 364, 73-75 (1993).

14. Plump A. S., Masucci-Magoulas L., Bruce C., Bisgaier C. L., Breslow J. L., & Tall A. R. Increased atherosclerosis in ApoE and LDL receptor gene knock-out mice as a result of human cholesteryl ester transfer protein transgene expression. Arterioscler Thromb Vasc Biol 19, 1105-110 (1999).

15. Hayek T. et al. Decreased early atherosclerotic lesions in hypertriglyceridemic mice expressing cholesterol ester transfer protein transgene. J Clin Invest 96, 2071-2074 (1995).

16. Lee R. T., & Libby P. The unstable atheroma. Arterios Throm Vasc Biol 17, 1859-1867 (1997).

17. Herrera V. L. M., Xie H. X., Lopez L. V., Schork N. J., & Ruiz-Opazo N. The 1 Na, K-ATPase gene is a susceptibility hypertension gene in the Dahl salt-sensitive rat. J Clin Invest 102, 1102-1111 (1998).

18. Lefer D. J. & Granger D. N. Monocyte rolling in early atherogenesis: vital role in lesion development. Circ Res 11, 1352-1355 (1999).

19. Nakashima Y., Raines E. W., Plump A. S., Breslow J. L., & Ross R. Upregulation of VCAM-1 and ICAM-1 at atherosclerosis-prone sites on the endothelium in the ApoE-deficient mouse. Arterioscler Thromb Vasc Biol 18, 842-851 (1998).

20. Nelken N., Couglin S., Gordon D., Wilcox J. Monocyte chemoattractant protein-1 in human atheromatous plaques. J Clin Invest 88, 1121-1127 (1991).

21. Shih D. M., Welch C., & Lusis A. J. New insights into atherosclerosis from studies with mouse models. Molecular Medicine Today (Elsevier Science Ltd.) 364-372 (1995).

22. Jiang X. C. et al. Down-regulation of messenger RNA for the low density lipoprotein receptor in transgenic mice containing the gene for human cholesteryl ester transfer protein. Mechanism to explain accumulation of lipoprotein B particles. J Biol Chem 268, 27406-27412 (1993).

23. Grass D. S. et al. Transgenic mice expressing both human apolipoprotein B and human CETP have a lipoprotein cholesterol distribution similar to that of normolipidemic humans. J Lipid Res 36, 1082-1091 (1995).

24. Hayek T. et al. An interaction between the human cholesteryl ester transfer protein (CETP) and apolipoprotein A-I genes in transgenic mice result in a profound CETP-mediated depression of high density cholesterol levels. J Clin Invest 90, 505-510 (1992).

25. Foger B., Ritsch A., Doblinger A., Wessels H., & Patsch J. R. Relationship of plasma cholesteryl ester transfer protein to HDL cholesterol: studies in normotriglyceridemia and moderate hypertriglyceridemia. Arterioscler Thromb Vasc Biol 16, 1430-1436 (1996).

26. Sechi L. A. et al. Glucose metabolism and insulin receptor binding and mRNA levels in tissues of Dahl hypertensive rats. Am J Hypertens 10, 1223-1230 (1997).

27. Kitagawa S., Yamaguchi Y., Shinozuka K, Kwon Y. M., & Kumitomo M. Dietary cholesterol enhances impaired endothelium-dependent relaxations in aortas of salt-induced hypertensive Dahl rats. Eur J Pharmacol 297, 71-76 (1995).

28. Reddick R. L., Zhang S. H. & Maeda N. Atherosclerosis in mice lacking apoE. Evaluation of lesional development and progression. Arterioscler Throm 14, 141-147 (1994).

29. Nakashima Y., Plump A. S., Raines E. W., Breslow J. L. & Ross R. ApoE-deficient mice develop lesions of all phases of atherosclerosis through the arterial tree. Arterioscler Throm 14, 133-140 (1994).

30. Ebara T., Ramakrishnan R, Steiner G, & Shacter N S. Chylomicronemia due to apolipoprotein CII overexpression in apolipoprotein E-null mice. J Clin Invest 99, 2672-2681 (1997).

31. Paigen B., Morrow A., Brandon C., Mitchell D., Holmes P. Variation in susceptibility to atherosclerosis among inbred strains of mice. Atherosclerosis 57, 65-73 (1985).

32. Ong G. L. & Mattes M. J. Mouse strains with typical mammalian levels of complement activity. J Immunol Methods 125, 147-158, 1991.

33. Torzewski K., Bowyer D. E., Waltenberger J., & Fizsimmons C. Processes in atherogenesis: complement activation. Atherosclerosis 132, 131-138, 1997.

34. Gotto A. M. Triglycerides and the development of atherosclerosis. In Atherosclerosis beyond cholesterol. Audio Visual Medical Marketing, Inc., New York, N.Y. pp 25-35, 1992.

35. Tropea B. I., Huie P., Cooke J. P., Tsao P. S., Sibley R. K., Zarins C. K. Hypertension-enhanced monocyte adhesion in experimental atherosclerosis. J Vasc Surg 23, 596-605 (1996).

36. Capers Q., et al. Monocyte chemoattractant protein-1 expression in aortic tissues of hypertensive rats. Hypertension 30, 1397-1402 (1997).

37. Shioi T. et al. Increased expression of interleukin-1 beta and monocyte chemotactic and activating factor/monocyte chemoattractant protein-1 in the hypertrophied and failing heart with pressure overload. Circ Res 81, 664-671 (1997).

38. O'Brien K. D. et al. Vascular cell adhesion molecule-1 is expressed in human coronary atherosclerotic plaques: implications for the mode of progression of advanced coronary atherosclerosis. J Clin Invest 92, 945-951 (1993).

39. Schecter A. D. et al. Tissue factor is induced by monocyte chemoattractant protein-1 in human aortic smooth muscle and THP-1 cells. J Biol Chem 272, 28568-28573 (1997).

40. Buja L. M., & Willerson J. T. Role of inflammation in coronary plaque disruption. Circulation 89, 503-505 (1994).

41. Boyle J. J. Association of coronary plaque rupture and atherosclerotic inflammation. J Pathology 181, 93-99 (1997).

42. Davies H, al-Tikriti S. Coronary arterial pathology in the transplanted heart. Int J Cardiol 25, 99-117 (1989).

43. Russell P. S., Chase O. M., Winn H. J. & Colvin R. B. Coronary atherosclerosis in transplanted mouse hearts. I. Time course and immounogenetic and immunopathological considerations. Am J Pathol 144, 260-274 (1994).

44. Atkinson J. B. Accelerated arteriosclerosis after transplantation: the possible role of calcium channel blockers. Int J Cardiol 62, Suppl 2:S125-134 (1997).

45. Drayna D. et al. Cloning and sequencing of human cholesteryl ester transfer protein cDNA, Nature 327, 623-634 (1987).

46. Zannis V. I., Cole S. F., Jackson C., Kurnit D. M., & Karathanasis S. K. Distribution of apo-A-I, apoC-II, apoC-III and apoE mRNA in human tissues. Time dependent induction of apoE mRNA by cultures of human monocyte-macrophages. Biochemistry 24, 4450-4455 (1985).

47. Adari H., Xiang X. H., Ruiz-Opazo N., Herrera V. L. M., & Makrides S. C. Functional validation of transgenes for the development of a transgene hypertensive rat atherosclerosis model. NATO Advanced Study Institute: “Vascular Endothelium: Pharmacologic and Genetic Manipulations.” Crete, Greece, Jun. 22-Jul. 1, 1996, in Vascular Endothelium: Pharmacologic and Genetic Manipulations (eds. J D Catravas, A D Callow, U S Ryan) 235-236 (NATO ASI Series, Plenum Press, New York, 1998).

48. Krauss R. M. Grunfeld C., Doerrler W. T., & Feingold K. R. Tumor necrosis factor acutely increases plasma levels of very low density lipoproteins of normal size and composition. Endocrinology 127, 1016-1021(1990).

TABLE 1
Comparative profile of lipid levels and lifespan on regular rat chow

| | | TC ± SEM | TG ± SEM | HDL_{c} ± SEM | TC/HDL_{c} ± SEM | Lifespan (N) |
|---|---|---|---|---|---|---|
| Line | CETP | [P value]* | [P value]* | [P value]* | [P value]* | [P value]** |
| control | bk | 142 ± 24 | 193 ± 35 | 59 ± 7.5 | 2.36 ± 0.13 | 29.3 ± 1.21 (24) |
| Tg21 | 131 | 146 ± 16 | 318 ± 50 | 46 ± 5.3 | 3.22 ± 0.20 | |
| | | | | | | 29.0 ± 2.80 (15) |
| Tg22 | 263 | 130 ± 6 | 245 ± 49 | 46 ± 4.7 | 2.93 ± 0.24 | |
| Tg25 | 2518 | 529 ± 92 | 3817 ± 254 | 37 ± 16 | 29.20 ± 7.8 | 27.86 ± 2.31 (7) |
| | | [<0.0006] | [<0.002] | | [<0.002] | |
| Tg53 | 3702 | 894 ± 98 | 6693 ± 1406 | 13 ± 6.1 | 137.1 ± 58 | 24.58 ± 2.20 (14) |
| | | [<10⁻¹] | [<0.02] | [<0.003] | [<0.02] | [<0.03] |
| Plasma lipid levels were measured at 6 months on regular rat chow after 24 hours fast in control Dahl S rats (n = 5); transgenic Tg21 (n = 5); Tg22 (n = 6); Tg25 (n = 7); Tg53 (n = 4) rats. Lifespan was determined on regular rat chow. CETP, | | | | | | |
| # cholesteryl ester transfer portein activity in units; HDL_{c}, mean high density lipoprotein cholesterol in mg/dl; (n), number of rats; SEM, standard error of the mean; [P value]*, t-test probability; [P value]** ANOVA; TC, mg/dl total cholestrol mean; TG, mg/dl triglyceride mean: control. littermate | | | | | | |
| # non-transgenic Dahl S male rats; bk. control CETP activity taken as background level. | | | | | | |

TABLE 2
Comparative analysis of lipid profiles at 10 weeks and 6 months of age:

| | TC | TG | VLDLc | VLDLtg | IDLc | IDLtg | LDLc | LDLtg | HDLc | HDLtg | ApoB |
|---|---|---|---|---|---|---|---|---|---|---|---|
| 10 weeks | | | | | | | | | | | |
| SS (−) | 113.6 | 80.6 | 4.0 | 33.1 | 4.3 | 19.77 | 36.7 | 14.2 | 63.7 | 13.5 | 15.3 |
| (n = 4) | (5.1) | (9.5) | (0.9) | (3.7) | (0.8) | (3.8) | (2.8) | (2.3) | (5.4) | (3.1) | (0.8) |
| Tg53 | 257.4*** | 2987.0*** | 225.6*** | 2679.0*** | 13.0* | 171.1** | 7.2*** | 59.7** | 6.2*** | 28.3* | 432.9*** |
| (n = 6) | (3.9) | (83.9) | (4.1) | (120.4) | (2.7) | (40.0) | (2.0) | (8.5) | (1.6) | (4.0) | (22.9) |
| Tg25 | 261.9*** | 963.9*** | 197.8*** | 870.9*** | 34.2** | 63.7* | 22.6* | 11.8^{N.S.} | 7.3*** | 17.4^{N.S.} | 147.1* |
| (n = 6) | (9.4) | (96.3) | (13.65) | (90.5) | (5.9) | (18.6) | (3.8) | (3.5) | (0.8) | (6.2) | (46.48) |
| Tg53 vs | ns | <0.0001 | ns | <0.0001 | 0.007 | 0.015 | 0.0045 | 0.0002 | ns | ns | 0.0001 |
| Tg25 | | | | | | | | | | | |
| 6 months | | | | | | | | | | | |
| SS (−) | 183.4 | 158.8 | 14.6 | 110.1 | 9.1 | 20.9 | 65.6 | 16.14 | 94.11 | 11.7 | 42.0 |
| (n = 8) | (12.0) | (12.14) | (1.6) | (10.9) | (1.8) | (4.1) | (4.9) | (3.3) | (6.7) | (1.5) | (6.7) |
| Tg53 | 544.7*** | 4410*** | 454.9*** | 3580*** | 49.8* | 638.4* | 17.0*** | 125.6*** | 21.4*** | 65.7** | 493.1*** |
| (n = 8) | (58.2) | (852.2) | (49.4) | (647.3) | (19.5) | (322.8) | (2.1) | (29.9) | (5.4) | (16.1) | (87.8) |
| Tg25 | 631.0*** | 5031*** | 586.4*** | 4726*** | 27.3*** | 214.5*** | 12.0*** | 68.0*** | 5.3*** | 22.3^{N.S.} | 643.1 |
| (n = 8) | (79.8) | (757.3) | (78.5) | (731.5) | (4.3) | (34.89) | (1.3) | (10.0) | (0.9) | (6.8) | (152.1) |
| Tg53 vs | n.s. | n.s. | n.s. | n.s. | n.s. | n.s. | 0.0295 | 0.0378 | 0.0052 | 0.0108 | n.s. |
| Tg25 | | | | | | | | | | | |

Legend: mean cholesterol and triglyeride values in mg/dl ± (standard error of the mean): TC, total plasma cholesterol; TG, total plasma triglyceride, VLDLc, cholesterol in VLDL; VLDLtg, triglyceride level in VLDL; ApoB, apolipoprotein B level. All are in mg/dl. *P < 0.05; **P < 0.001; ***P < 0.0001.

What is claimed is:

1. A non-human transgenic mammal that exhibits an atherosclerosis phenotype as a consequence of the expression of a transgene encoding mammalian cholesteryl ester transfer protein (CETP), wherein said mammal, in its non-transgenic form, is salt-sensitive such that it becomes hypertensive on a diet containing a higher than normal salt content, and wherein said CETP transgene is expressed under the control of a promoter that causes the transgene to be expressed in the liver of said mammal.

2. The mammal of claim 1, wherein said mammal is a rodent.

3. The rodent of claim 2, wherein said rodent is a rat.

4. The mammal of claim 1, wherein said CETP gene is the human CETP (hCETP) gene.

5. The mammal of claim 1, wherein the promoter is the ApoC3 promoter.

6. The rat of claim 3, wherein the rat is a Dahl S rat.

7. The rat of claim 6, wherein said transgene is hCETP.

8. The rat of claim 7, wherein said promoter is the ApoC3 promoter.

9. A method for testing a compound for its potential efficacy in treating or slowing the development of heart disease, said method comprising administering said compound to the mammal of claim 1, under conditions under which said mammal would, in the absence of treatment, develop atherosclerosis and hypertension, and determining whether such development is slowed or reversed by said compound.

10. A method for diagnosing a human patient with early-stage heart disease, said method comprising the steps of:
(a) determining one or more early-stage indicators of cardiac disease in a mammal of claim 1,
(b) measuring or observing in said human patient at least one of the counterparts of said one or more early stage indicators; and
(c) diagnosing said human with early stage heart disease when one or more of the indicators of (a) are measured or observed in (b).
